(19) United States
(12) Patent Application Publication
(10) Pub. No.: US 2006/0039984 A1
(43) Pub. Date: Feb. 23, 2006

(54) Process for producing fine porous polyimide particle

(76) Inventors: Hachiro Nakanishi, Sendai-shi (JP); Hitoshi Kasai, Sendai-shi (JP); Hirohiko Miura, Sendai-shi (JP); Hidetoshi Oikawa, Sendai-shi (JP); Shuji Okada, Sendai-shi (JP)

Correspondence Address: SHERMAN & ASSOCIATES, 415 NORTH ALFRED STREET, ALEXANDRIA, VA, 22314, US

(21) Appl. No.: 10/537,497

(22) PCT Filed: Jul. 9, 2003
(86) PCT No.: PCT/JP03/08728

§ 371 Date: Jun. 3, 2005

(30) Foreign Application Priority Data
Dec. 16, 2002 (JP) 2002-363973

(51) Int. Cl. A61K 9/14; B29B 9/00

(52) U.S. Cl. 424/489; 264/5

(57) ABSTRACT

A method for preparation of porous polyimide microparticles comprising, forming polyamide acid microparticles by pouring polymer solution prepared by dissolving polyamide acid containing 0.5 to 80 weight % of alkali metal salt to polyamide acid by 0.1 to 15 weight % concentration into a poor solvent selected from the group consisting of aliphatic solvents, alicyclic solvents, aromatic solvents, CS₂ and mixture of two or more these solvents and the temperature of which is adjusted to the range from −20° C. to 60° C., wherein particle size of said polyamide acid microparticles is adjusted to 50 nm to 10000 nm by controlling the temperature of said poor solvent, pore size of said polyamide acid microparticles is adjusted to the range from 20 nm to 500 nm and porosity of said polyamide acid microparticles is adjusted to the range from 0.1% to 30% by controlling a content or a kind of said alkali metal salt, then treating said polyamide acid microparticles by chemical imidation or thermal imidation, or by thermal imidation after chemical imidation so that the particle size distribution, pore size and porosity of said polyamide acid microparticles can be maintained.

### FIELD OF THE INVENTION

The present invention relates to a method for preparation of porous polyimide microparticles with good reproducibility by pouring polyamide acid solution containing prescribed amount of alkali metal salt into a poor solution, forming porous polyamide acid microparticles by reprecipitation method then imidate said porous polyamide acid microparticles.

In the present invention, the term of porosity indicates the ratio of total surface area of pores to the surface area of a microparticle.

### DESCRIPTION OF THE PRIOR ART

Porous polyimide microparticles have various uses such as catalyst carrier, drug delivery, separation filter or low dielectric materials, because said particles have excellent heat-resistance, solvent-resistance and good insulating ability. Further, regarding the polyimide microparticle, the formed fine pores are characterized as nano meter size level structure and is useful as a template for various materials of nano meter size level particle size.

Generally, for the preparation of porous polymer materials, for example, a spray dray method is used. However, this method has a problem that the controlling of preparation condition and the selection of solvent are very difficult. Therefore, porous polyimide microparticles which can be more simply prepared and has good reprecipitation, especially is excellent in chemical and physical properties are desired.

Document; Hyuck Jai Lee, Jongok Won, Hoosung Lee, Yong Soo Kang, “Solution properties of poly(amic acid)-NMP containing LiCl and their effects on membrane morphologies” Journal of Membrane Science 196, 267-277 (2002) (document 1). In said document 1, following subjects are described, that is, adding of LiCl to poly(amic acid)-NMP solution effects to properties of solution and shape of a membrane to be formed, a membrane formed by casting using poly(amic acid)-NMP solution containing LiCl is difficult to form large pore because the polymer has a tendency to be gelled and mainly forms spongy structure, further, the increase of LiCl concentration causes the increase of porosity of the membrane.

In the meanwhile, the inventors of the present invention have established a method to form nano meter size particles of polyimide, that is, after preparation of polyamide acid microparticles whose size is controlled to nano meter order by a reprecipitation method characterizing to pour polyamide acid solution into poor solution having compatibility with a solvent of said solution and carry out imidation treatment (Polymer Society Annual Convention held on May 25, 2001; investigation Report, titled “IIIFO 8 Preparation of Polyimide Microparticles by Reprecipitation Method”, Polymer Preprint, Japan Vol. 50, No. 3, p484, document 2). In the formation of polyimide nano meter size particles by said reprecipitation method, sometimes there is a case to form porous polyimide nano meter size particles, however, the reproductivity of it is bad.

Document; X. D. ZHAU, S. C. ZHANG, W. HUEBNER, P. D. OWNBY, “Effect of the solvent on the particle morphology of spray dried PMMA”, JOURNAL OF MATERIALS SCIENCE, Vol. 36, 3759-3767 (2001) (document 3). In said document, the following subjects are reported that, in a spray dry method, as the solvent of PMMA following two combination cases are reported, that is, one is the combination of acetone/water and another one is the combination of THF/water. In farmer case, porous particles are formed and in latter case, particles of honey comb structure are formed, and reporting that this difference is based on the difference of mutual function of solvent and PMMA. That is, the authors refer that the shape of formed particles is effected by solvent to be used and others.

The subject of the present invention is to provide a method for preparation of porous microparticles polymer, especially porous polyimide microparticles further especially of nano meter size which is characterized to be simple and with good reproductivity and from which defects of said spray dry method are removed. For the dissolution of said subject, the inventors of the present invention have continued earnest investigation to apply the method for preparation of polyimide microparticles, especially nano size particles developed by the inventors of the present invention. In the investigation, the inventors of the present invention have tried a reprecipitation method as follows, that is, after preparing polyamide acid pouring dianhydride of 2,2-(3,4-dicarboxyphenyl)-1,1,1,3,3,3-hexafluoro propane and 4,4-diaminodiphenyl ether into N-methylpyrrolidone (NMP), polyamide acid solution in which prescribed amount of LiCl is blended is prepared, then said polyamide acid solution is poured into cyclohexane, which is a poor solvent of said polyamide acid or mixture of cyclohexane to which CS₂ is added, and the inventors of the present invention have found that the porous (physical properties such as pore size and porosity) polyamide acid corresponding to the blending amount of LiCl is formed, and said porous polyamide acid can be converted to porous polyimide microparticles by chemical imidation, and dissolved said subjects.

Further, in the case when a chloride ion is changed to other halogen ion or other anion such as nitric ion, it is confirmed that similarly porous polyimide can be formed. In particular, it is confirmed that LiBr indicates same characteristic to LiCl. Further, in the case when lithium ion is changed to other alkali metal ion, it is confirmed that porous polyimide can be formed.

### SUMMARY OF THE INVENTION

The present invention is (1) a method for preparation of porous polyimide microparticles comprising, forming polyamide acid microparticles by pouring polymer solution prepared by dissolving polyamide acid containing 0.5 to 80 weight % of alkali metal salt to polyamide acid by 0.1 to 15 weight % concentration into a poor solvent selected from the group consisting of aliphatic solvents, alicyclic solvents, aromatic solvents, CS₂ and mixture of two or more these solvents and the temperature of which is adjusted to the range from −20° C. to 60° C., wherein particle size of said polyamide acid microparticles is adjusted to 50 nm to 10000 nm by controlling the temperature of said poor solvent, pore size of said polyamide acid microparticles is adjusted to the range from 20 nm to 500 nm and porosity of said polyamide acid microparticles is adjusted to the range from 0.1% to 30% by controlling a content or a kind of said alkali metal salt, then treating said polyamide acid microparticles by chemical imidation or thermal imidation, or by thermal imidation after chemical imidation so that the particle size distribution, pore size and porosity of said polyamide acid microparticles are maintained.

Desirably, the present invention is (2) the method for preparation of polyimide microparticles, after forming polyamide acid microparticles having particle size distribution, pore size and porosity of said (1), then treating said polyamide acid microparticles by imidation so that the particle size distribution, pore size and porosity of said polyamide acid microparticles are maintained, wherein organic solvent used as the good solvent is at least one selected from the group consisting of N,N-dimethylacetoamide, dimethylformamide, N-methylpyrrolidone (NMP), which are polar amide solvents or mixture of two or more these solvents, and poor solvent is at least one selected from the group consisting of decalin, cyclohexane, hexane, benzene, toluene, CS₂ or mixture of two or more these solvents, more desirably, the present invention is (3) the method for preparation of polyimide microparticles of (1) or (2), wherein chemical imidation is comprising, completing by adding acetic acid anhydride-pyridine mixed solvent to polyamide acid microparticles dispersion having pore size and porosity controlled by a content or a kind of alkali metal salt by stirring, further desirably, the present invention is (4) the method for preparation of polyimide microparticles of (1), (2) or (3), wherein average molecule weight (weight average) is from 800 to 220000.

### BRIEF DESCRIPTION OF THE DRAWINGS

FIG. 1 shows a concept of a preparation process of porous polyimide microparticles of the present invention.

FIG. 2 shows SEM picture of porous polyimide microparticles obtained by preparing a solution so as the blending ratio of LiCl to polyamide acid (PAA) of Example 1 to be respectively 5 mass % (a), 10 mass % (b) and 20 mass % (c).

FIG. 3 shows SEM picture of porous polyimide microparticles obtained by pouring solution of polyamide acid to which LiCl of 20 weight % is added to cyclohexane mixture to which 10 volume % (a) or 20 volume % of CS₂ of Example 2 is added.

FIG. 4 shows SEM picture of porous polyimide microparticles obtained by preparing a solution so as the blending ratio of LiBr (1), LiI (b), LiNO₃ (c) to be 20 mass % to polyamide acid using the to polyamede acid solution of Example 3.

FIG. 5 shows SEM picture of porous polyimide microparticles obtained by injecting solution of polyamide acid of Example 6 to which LiCl is blended so as the blending amount to be 20 mass % polyamide acid is added into cyclohexane at 20° C. (a), 40° C. (b) and 60° C. (c) using a micro syringe, under stirring condition of 1500 rpm.

### DESCRIPTION OF THE PREFERRED EMBOBYMENT

The present invention will be illustrated more in detail.

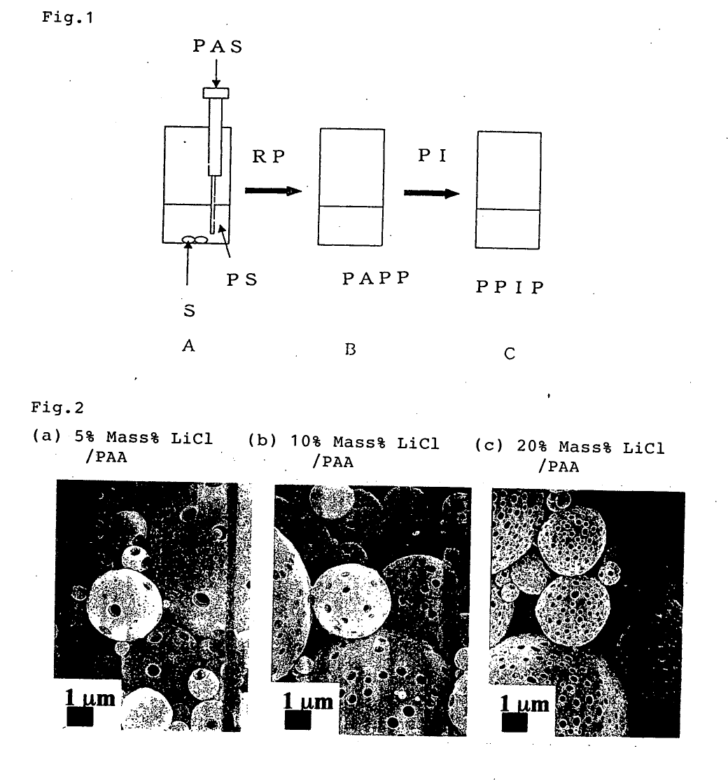

A. Reprecipitation method to form porous polyamide acid microparticles used in the present invention is not different from the method used for the preparation of polyamide acid microparticles except containing alkali metal salt in solution. Process of reprecipitation is shown in FIG. 1. In processes A and B, solution of polyamide acid PAS containing prescribed amount of alkali metal salt is poured into poor solvent PS, and porous polyamide acid microparticles PAPP having pore size and porosity corresponding to a content or a kind of alkali metal salt by reprecipitation method RS. Stirring of the poor solvent at the injection of solution is desirable to be carried out by rotating a stirrer S by 100-3000 rpm, however, not necessarily to be used. For the purpose to improve dispersability of prepared porous microparticles, it is possible to contain 0.1 weight % of a neutral polymer surface active detergent (Acrydic: products of Dainihon Ink Co., Ltd.), which is a kind of polyacrylic acid ester, however, this is not necessarily to be used. Then, acetic acid anhydride/pyridine mixed solvent is added at C process and chemically imidated PI under stirring by 100-3000 rpm, and polyimide microparticles PPIP which maintains the pore size and porosity of above mentioned porous polyamide acid microparticles. Thermal imidation can be used as an imidation process, or thermal imidation can be carried out after chemical imidation.

B. As the solvent for polyamide acid (can be called as polyamic acid), conventional organic solvent can be used, and as the example, acetone, methylethyl ketone, tetrahidrofurane, dioxane, acetonitlile, alcohols (methanol, ethanol, isopropanol etc), N,N-dimethylacetoamide, demethylformamide or N-methylpyrrolidone (NMP) can be mentioned, among these compounds, N,N-dimethylacetoamide, NMP or demethylformamide, which are polar amide solvents are desirably used. Concentration of polyamide acid solution is a large factor which effects to particle size, in particular, larger molecule weight of polyamide acid promises larger effect of concentration of solution. Desirable concentration of polyamide is from 0.1 to 15.0 weight %, and when molecular weight is large desirable concentration is around 0.5 weight %. Further, when the concentration becomes high, specifically 4.0 weight %, porous microparticles are flocculated.

C. As an example of poor solvent for said polyamide acid having good compatibility with a solvent of the polyamide acid, hexane (aliphatic), decalin, -cyclohexane (alicyclic), benzene, toluene (aromatic) or carbon disulfide or mixture of two or more these compounds can be used. Among these solvents, alicyclic solvents and mixed solvent consisted of alicyclic solvents and carbon disulfide are desirably used.

D. Temperature of the poor solvent is sufficient to be room temperature, however, since it is possible to prepare porous polyamide acid microparticles whose particle size is controlled by controlling temperature consition, and in the case of temperature lower than 30° C., tendency that the particle size of porous polyamide acid microparticles becomes large is observed, and the porous polyamide acid microparticles whose size is 10000 nm by maximum is formed.

E. Basically, molecular weight of polymide used in the present invention can be voluntarily selected by relation with an use of porous polyimide microparticles, however, for the stable preparation of microparticles having desired particle size, it is desirable that the average molecular weight (weight) is in the range from 8000 to 220000.

### EXAMPLE

The present invention will be illustrated more in detail, however, not intending to limit the scope of the claims of the present invention.

### Example 1

Polyamide acid (molecular weight: 68650) obtained by polymerization of 2,2-(3,4-dicarboxyphenyl)-1,1,1,3,3,3-hexafluoropropane dianhydride and 4,4-diaminodiphenyl dissolved in NMP (afore mentioned Acrydic: containing 0.1 weight %) by 1.54 weight %, then solutions are prepared so as the blending amount of LiCl to polyamide acid contained in polyamide solution to be 5 mass % (a), 10 mass % (b) and 20 mass % (c)/polyamide acid. 0.1 ml of said prepared solutions are respectively poured into 10 ml of cyclohexane by a microsyringe under stirring condition of 1500 rpm at room temperature. Porosity of formed porous polyamide acid microparticles becomes larger along with the increase of blending amount of LiCl.

0.1 mL of mixed solution of pyridine/acetic acid anhydride whose molar ratio is 1/1 is added to above porous polyamide acid microparticles dispersion by stirring then continue the stirring for about 2 hours, thus chemical imidation is completed. Porous polyimide microparticles maintaining porous properties of above mentioned porous polyamide acid microparticles are obtained. Porous properties (pore size, particle size and porosity) are observed by a Scanning Electro Microscope. Results are summarized in FIG. 2.

### Example 2

Polyamide acid (molecular weight: 68650) obtained by polymerization of 2,2-(3,4-dicarboxyphenyl)-1,1,1,3,3,3-hexafluoropropane dianhydride and 4,4-diaminodiphenyl dissolved in NMP (afore mentioned Acrydic: containing 0.1 weight %) by 1.54 weight %, then LiCl of 20 weight % is added to polyamide acid. 0.1 ml of said obtained solution is poured into cyclohexane mixture (10 ml) to which 10 vol % (a) or 20 vol % CS₂ is added using a micro syringe at room temperature under stirring condition by 1000 rpm. Particle size and porosity of formed porous polyamide acid microparticles become small along with the increase of blending amount of CS₂.

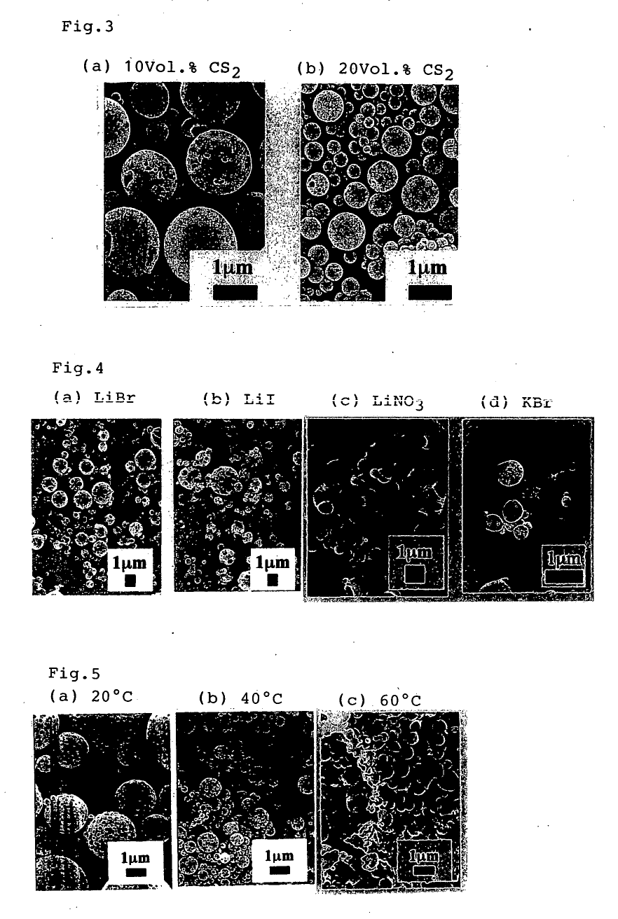

To the obtained porous polyamide acid microparticles dispersion, 0.1 ml of mixed solution of pyridine/acetic acid anhydride by 1/1 molar ratio is added with constant stirring, that is, chemical imidation to maintain said condition for about 2 hours is carried out, then thermal imidation to maintain the temperature of 250° C. for 3 hours is carried out. Consequently, porous polyimide microparticles characterized that the imidation is quantitatively progressed, maintaining porous properties of the porous polyamide acid microparticles, is obtained. Porous properties (pore size, particle size and porosity) are observed by a Scanning Electro Microscope. Results are summarized in FIG. 3.

### Example 3

Polyamide acid (molecular weight: 68650) obtained by polymerization of 2,2-(3,4-dicarboxyphenyl)-1,1,1,3,3,3-hexafluoropropane dianhydride and 4,4-diaminodiphenyl dissolved in NMP (afore mentioned Acrydic: containing 0.1 weight %) by 1.54 weight %, then solutions are prepared so as the blending amount of LiBr (a), LiI (b), LiNO₃ (c) and KBr (d) to polyamide acid contained in polyamide acid solution to be 20 mass %/polyamide acid. 0.1 ml of the obtained solutions are respectively poured into 10 ml of cyclohexane using a micro syringe at room temperature under stirring condition by 1500 rpm. Although pore size or porosity of formed porous polyamide acid microparticles are different according to the kind of the alkali metal salt, formation of porous polyamide acid microparticles is observed in all specimen. In the case of use of LiNO₃, maximum pore size becomes around 500 nm.

To said porous polyimide microparticles dispersion, 0.1 ml of mixed solution of pyridine/acetic acid anhydride by 1/1 molar ratio is added with constant stirring, that is, chemical imidation to maintain said condition for about 2 hours is completed and porous polyimide acid micropartides maintaining porous properties of the porous polyamide acid microparticles are obtained. Porous properties (pore size, particle size and porosity) are observed by a Scanninng ELectro Microscope. Results are summarized in FIG. 4.

### Example 4

Polyamide acids having various molecular weights: (8000, 48000, 69000, 93000, 220000) obtained by polymerization of 2,2-(3,4-dicarboxyphenyl)-1,1,1,3,3,3-hexafluoropropane dianhydride and 4,4-diaminodiphenyl dissolved in NMP (afore mentioned Acrydic: containing 0.1 weight %) by 1.54 weight %, then solutions are prepared so as the blending amount of LiCl to polyamide acid contained in polyamide solution to be 20 mass %/polyamide acid. 0.1 ml of said prepared solutions are respectively poured into 10 ml of cyclohexane by a microsyringe under stirring condition of 1500 rpm at room temperature.

To said porous polyimide microparticles dispersion, 0.1 ml of mixed solution of pyridine/acetic acid anhydride by 1/1 molar ratio is added with constant stirring, that is, chemical imidation to maintain said condition for 2 hours around is completed and porous polyimide acid microparticles maintaining porous properties of the porous polyamide acid microparticles are obtained. Porous properties (pore size, particle size and porosity) are observed by a Scanning Electro Microscope. The tendency that particle size and porosity becomes small when the average molecular weight of polyamide acid to be used is small can be observed.

### Example 5

Polyamide acid (molecular weight: 120000) obtained by polymerization of pyromellitic acid dianhydride and 4,4-oxydianilin is dissolved in NMP/DMF (1:1 mixture) by 0.1-15.0 weight % and prepare a solution so as the blending amount of LiCl to polyamide acid contained in polyamide acid solution to be 20 mass %/polyamide acid, then 0.1 ml of the prepared solution is poured into 10ml of cyclohexane using a microsyringe under stirring condition by 1500 rpm at room temperature.

To said porous polyimide microparticles dispersion, 0.1 ml of mixed solution of pyridine/acetic acid anhydride by 1/1 molar ratio is added with constant stirring, that is, chemical imidation to maintain said condition for about 2 hours is completed and porous polyimide microparticles, which maintains porous properties of the porous polyamide acid microparticles, are obtained. Porous properties (pore size, particle size and porosity) are observed by a Scanning Electro Microscope. The tendency that particle size and porosity become small when the concentration of polyamide acid solution to be used is low. And when the concentration of polyamide acid solution exceeds 4.0 weight %, the porous microparticles are flocculated.

### Example 6

Polyamide acid (molecular weight: 68650) obtained by polymerization of 2,2-(3,4-dicarboxyphenyl)-1,1,1,3,3,3-hexafluoropropane dianhydride and 4,4-diaminodiphenyl is dissolved in NMP (afore mentioned Acrydic: containing 0.1 weight %) by 1.54 weight %, then solution is prepared so as the blending amount of LiCl to polyamide acid contained in polyamide solution to be 20 mass %/polyamide acid. 0.1 ml of said prepared solution is respectively poured into 10 ml of cyclohexane using a microsyringe under stirring condition of 1500 rpm at 20° C. (a) and 40° C. (b). Further, by same condition as mentioned above, 0.1 ml of the solution whose blending ratio of LiCl to polyamide acid is 60 mass % is added to 10 ml of cyclohexane at 60° C. (c). When the porous polyamide acid microparticles are prepared by higher temperature, particle size and pore size become smaller, and minimum values are respectively around 50 nm and 20 nm.

To said porous polyimide microparticles dispersion, 0.1 ml of mixed solution of pyridine/acetic acid anhydride by 1/1 molar ratio is added with constant stirring, that is, chemical imidation to maintain said condition for around 2 hours is completed and porous polyimide microparticles, which maintains porous properties of the porous polyamide acid microparticles, are obtained. Porous properties (pore size, particle size and porosity) are observed by a Scanning Electro Microscope. Results are shown in FIG. 5.

### INDUSTRIAL APPLICABILITY

The method of the present invention is to provide an art which has high possibility for industrial applicability promising to prepare porous polyimide microparticles which meets to the purpose for use easily with good re-productivity, controlling pore size, porosity and particle size by using an established reprecipitation method to prepare polyimide microparticles.

1. A method for preparation of porous polyimide microparticles comprising, forming polyamide acid microparticles by pouring polymer solution prepared by dissolving polyamide acid containing 0.5 to 80 weight % of alkali metal salt to polyamide acid by 0.1 to 15 weight % concentration into a poor solvent selected from the group consisting of aliphatic solvents, alicyclic solvents, aromatic solvents, CS₂ and mixture of two or more these solvents and the temperature of which is adjusted to the range from −20° C. to 60° C., wherein particle size of said polyamide acid microparticles is adjusted to 50 nm to 10000 nm by controlling the temperature of said poor solvent, pore size of said polyamide acid microparticles is adjusted to the range from 20 nm to 500 nm and porosity of said polyamide acid microparticles is adjusted to the range from 0.1% to 30% by controlling a content or a kind of said alkali metal salt, then treating said polyamide acid microparticles by chemical imidation or thermal imidation, or by thermal imidation after chemical imidation so that the particle size distribution, pore size and porosity of said polyamide acid microparticles are maintained.

2. The method for preparation of polyimide microparticles, after forming polyamide acid microparticles having particle size distribution, pore size and porosity of claim 1, then treating said polyamide acid microparticles by imidation so that the particle size distribution, pore size and porosity of said polyamide acid microparticles are maintained, wherein organic solvent used as the good solvent is at least one selected from the group consisting of N,N-dimethylacetoamide, dimethylformamide, N-methylpyrrolidone (NMP), which are polar amide solvents or mixture of two or more these solvents, and poor solvent is at least one selected from the group consisting of decalin, cyclohexane, hexane, benzene, toluene, CS₂ or mixture of two or more these solvents.

3. The method for preparation of polyimide microparticles of claim 1, wherein chemical imidation is comprising, completing by adding acetic acid anhydride-pyridine mixed solvent to polyamide acid microparticles dispersion having pore size and porosity controlled by contents or kinds of alkali metal salt by stirring.

4. The method for preparation of polyimide microparticles, after forming polyamide acid microparticles having particle size distribution, pore size and porosity of claim 1, then treating said polyamide acid microparticles by imidation so that the particle size distribution, pore size and porosity of said polyamide acid microparticles can be maintained, wherein organic solvent used as the good solvent is at least one selected from the group consisting of N,N-dimethylacetoamide, dimethylformamide, N-methylpyrrolidone (NMP), which are polar amide solvents or mixture of two or more these solvents, and poor solvent is at least one selected from the group consisting of decalin, cyclohexane, hexane, benzene, toluene, CS₂ or mixture of two or more these solvents, and chemical imidation is comprising, completing by adding acetic acid anhydride-pyridine mixed solvent to polyamide acid microparticles dispersion having pore size and porosity controlled by contents or kinds of alkali metal salt by stirring.

5. The method for preparation of polyimide microparticles of claim 1, wherein weight average molecular weight of polyamide acid is in the range from 8000 to 220000.

6. The method for preparation of polyimide microparticles, after forming polyamide acid microparticles having particle size distribution, pore size and porosity of claim 1, then treating said polyamide acid microparticles by imidation so that the particle size distribution, pore size and porosity of said polyamide acid microparticles are maintained, wherein organic solvent used as the good solvent is at least one selected from the group consisting of N,N-dimethylacetoamide, dimethylformamide, N-methylpyrrolidone (NMP), which are polar amide solvents or mixture of two or more these solvents, and poor solvent is at least one selected from the group consisting of decalin, cyclohexane, hexane, benzene, toluene, CS₂ or mixture of two or more these solvents and weight average molecular weight of polyamide acid is in the range from 8000 to 220000.

7. The method for preparation of polyimide microparticles, after forming polyamide acid microparticles having particle size distribution, pore size and porosity of claim 1, then treating said polyamide acid microparticles by imidation so that the particle size distribution, pore size and porosity of said polyamide acid microparticles can be maintained, wherein organic solvent used as the good solvent is at least one selected from the group consisting of N,N-dimethylacetoamide, dimethylformamide, N-methylpyrrolidone (NMP), which are polar amide solvents or mixture of two or more these solvents, and poor solvent is at least one selected from the group consisting of decalin, cyclohexane, hexane, benzene, toluene, CS₂ or mixture of two or more these solvents, weight average molecular weight of polyamide acid is in the range from 8000 to 220000 and chemical imidation is comprising, completing by adding acetic acid anhydride-pyridine mixed solvent to polyamide acid microparticles dispersion having pore size and porosity controlled by contents or kinds of alkali metal salt by stirring.
